(19) United States
(12) Plant Patent Application Publication
(10) Pub. No.: US 2014/0041087 P1
(43) Pub. Date: Feb. 6, 2014

(54) BLACKBERRY PLANT NAMED 'EMILIA'

(50) ;

(71) Applicants: Maria Pilar Bañados Ortiz, San Fernando (CL); Alejandra Andrea Salgado Rojas, Santiago (CL); Jessica Maria González Mahan, Santiago (CL); John Reuben Clark, Fayetteville, AR (US)

(72) Inventors: Maria Pilar Bañados Ortiz, San Fernando (CL); Alejandra Andrea Salgado Rojas, Santiago (CL); Jessica Maria González Mahan, Santiago (CL); John Reuben Clark, Fayetteville, AR (US)

(73) Assignee: Hortifrut S.A., Estero, FL (US)

(21) Appl. No.: 13/568,018

(22) Filed: Aug. 6, 2012

(51) Int. Cl. A01H 5/00

(52) U.S. Cl. PLT/203

(57) ABSTRACT

A new and distinct cultivar of Blackberry plant named ‘Emilia’ as described and shown herein. Distinctive characteristics of ‘Emilia’ include its earliness, thornless stems, large sized fruit and sweetness without bitter aftertaste.

### BACKGROUND AND SUMMARY

Blackberries are a well-known, aggregate fruit enjoyed by many throughout the world. One example of an existing blackberry variety is ‘Tupy’. Another example of an existing, patented blackberry variety is ‘Navajo’, U.S. Plant Pat. No. 6,679.

Compared to Tupy, the present cultivar, ‘Emilia’, does not have spines, and the fruits are smaller than Tupy (6.1 grams versus 8.0 grams, average). However, the fruits of Emilia are longer (2.8 versus 2.4 cm) than Tupy. In addition, while the acidity and sugar content of the fruits are similar, Emilia does not have the bitter aftertaste that Tupy has. The fruits of Emilia ripen about one week before Tupy.

Compared to Navajo, the present cultivar, ‘Emilia’, has vegetative vigor much higher than Navaho. In the experimental garden in Nogales, Fifth Region, Chile, where both varieties were grown and evaluated, the fruit of Emilia was much larger (6.1 g v/s 3.0 g) and longer (2.8cm v/s 1.5 cm). Emilia's fruit has more acidity and less soluble solids than Navaho. Emilia ripens 5 weeks before Navaho.

Distinctive characteristics of ‘Emilia’ include its earliness, thornless stems, large sized fruit and sweetness without bitter aftertaste.

### BRIEF DESCRIPTION OF THE PHOTOGRAPHS

FIG. 1 is a photograph of a primocane of the Blackberry cultivar ‘Emilia’, showing the purple coloration and absence of thorniness of the stems. This picture was taken three months after primocane emergence, but the section of the primocane shown is about one month's growth (photo taken 9 Feb. 2012).

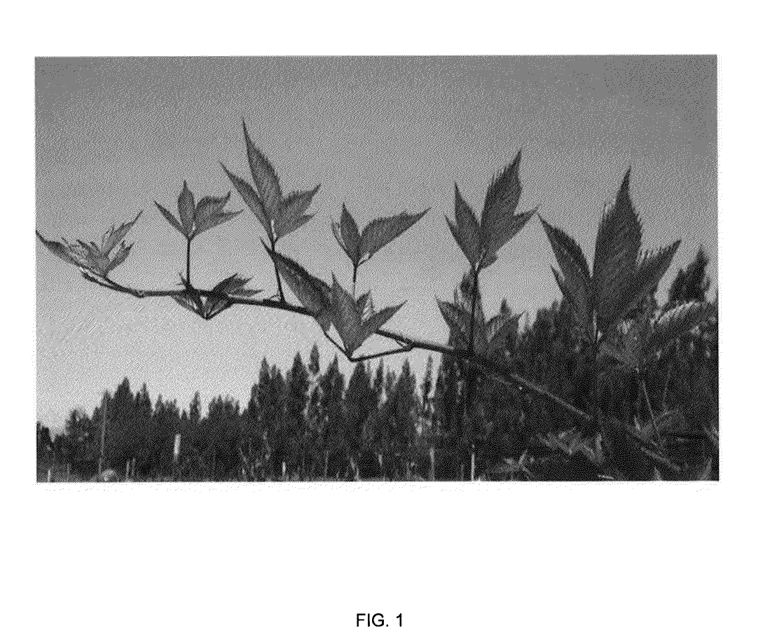

FIG. 2 is a photograph of fruiting laterals, this picture demonstrates the high productivity of the Blackberry cultivar ‘Emilia’. This picture was taken on 12 Dec. 2011 and shows floricanes that are about 15 months old at the time of the photo (note: the floricanes had been pruned in January 2011 at about 1.2 m height in order to stimulate the breaking of the laterals. (Eucalyptus globulus trees in the background.)

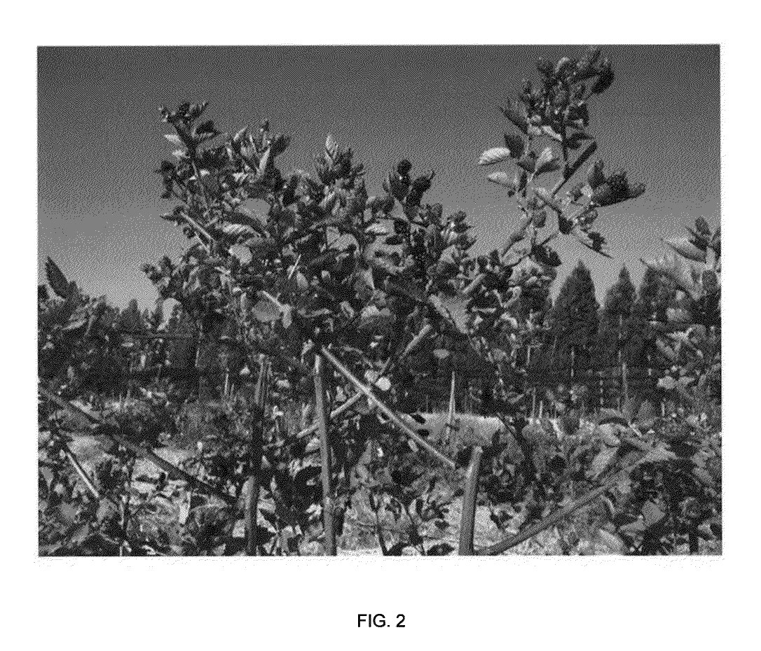

FIG. 3 is a close-up photograph of ripe and unripe fruit on floricanes of the Blackberry cultivar ‘Emilia’. Photograph taken 12 Dec. 2011.

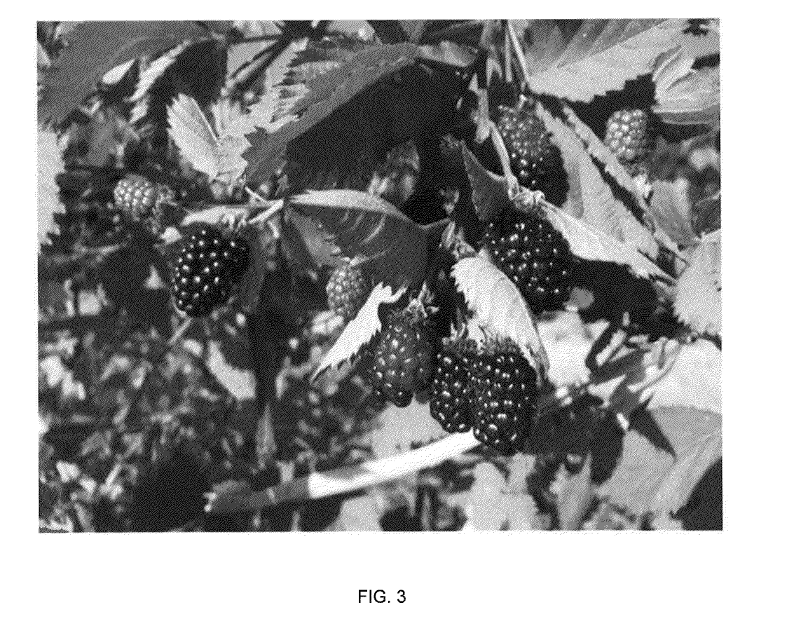

### DETAILED DESCRIPTION

Note: statements of characteristics herein represent exemplary observations of the cultivar herein and will vary depending on time of year, location, annual weather, etc. Where dimensions, sizes, colors, and other characteristics are given, it is to be understood that such characteristics are approximations and averages. The descriptions reported herein are from specimen plants that were planted in August 2009 at Nogales, Fifth Region, Chile.
Cultivar name: ‘Emilia’
Classification:
   Family.—Rosaceae.
   Botanical name.—Rubus subgenus Rubus.
   Common name.—Blackberry.
Parentage:
   Female parent.—Name: A-2240T. This was a proprietary breeding line that was never patented and was discarded in 2008 (i.e. it doesn't exist anymore). The cane habit of A-2240T was erect to semi-erect and it ripened in the early season, near Arapaho (U.S. Plant Pat. No. 8,510) and Natchez (U.S. Plant Pat. No. 20,891). A-2240T, therefore, likely would have ripened somewhat earlier than Emilia had they been compared in the same site. A-2240T had long, heavy berries, weighing about 8 g. A-2240T was a sibling of Natchez.
   Male parent.—Name: A-1689 T. This was a proprietary breeding line that was never patented and that is about to be discarded (i.e. it will soon not exist anymore). A-1689T ripens much later than Emilia, mid-late season, near Navaho (U.S. Plant Pat. No. 6,679) and Apache (U.S. Plant Pat. No. 11,865). A-1689T has large berries, 7-8 g, that are medium elongated but not extended long shape. The cross between the parents was made near Clarksville, Arkansas, USA in 2005 and resulted from a controlled hand pollination of female parent A-2240T x A-1689T male parent. Seeds from this controlled pollination were then sent to a nursery at Hijuelas, Fifth Region, Chile in September 2005. These seeds were germinated in a greenhouse between November 2005 and March 2006 and planted into the selection field at the experimental garden at Nogales, Fifth Region, Chile in December 2006. The seedlings fruited for the first time in the southern hemisphere summer of 2006/2007 on floricanes and one seedling, designated HFM-7 and now known as “Emilia”, was selected on 11 Dec. 2007 for having early fruits that were large, sweet, and without noticeable aftertaste, and the plant was also thornless. Emilia was first asexually propagated by taking root cuttings from the original plant which was growing in a field in Nogales, Fifth Region, Chile. Emilia was dug-up completely and asexually propagated from the root pieces and by dividing the crown of the plant into individual canes. Emilia has also been put into in vitro culture in Macul, Santiago, Chile. The growing location for many of the observations herein was Nogales, Fifth Region, Chile, in about 2007-2012. The date of the many of the observations was Dec. 11, 2007; other observations were obtained over two fruiting cycles in Chilean summer 2010/2011 and summer 2011/2012. Yield, overall plant health and vigor, fruit quality (at harvest and post-harvest) were further evaluated and informal taste tests were performed. Emilia was above average on all characteristics evaluated and ranked among the top three of ten genotypes in taste tests.
General description: Plants of Emilia have high vegetative vigor, similar to the variety Tupy. The canes are completely thornless, have a green background color with a prominent reddish blush where exposed to light, erect in habit, and only produce flowers and fruits on previous-year's canes (i.e. floricane fruiting). Emilia is a productive variety, similar to Tupy in yield, the fruits are smaller than Tupy, with good flavor, high firmness, with almost no color regression (druplets turning from black to red) in cold storage, and the fruits have very little leakage of juice in post-harvest, which indicates that they are firm and will resist post-harvest decay.
Growth: Plants have high vegetative vigor, erect growth habit. Primocanes emerge both from the crown of the plant as well as from the roots (as suckers).
Growth rate: The growth rate is rapid, with canes reaching 1.5 meters in heights within one month of emergence from the soil in the spring.
Productivity: High
Cold hardiness: Ultimate cold hardiness is unknown, but in Chile dormant plants have resisted midwinter lows of −7° C. without damage.
Branching height of the plants: Unknown (they are always pinched to induce branching and never left to grow to their own devices).
Canes:
General description: Thornless, vigorous, very erect, thick diameter relative to other varieties (Navaho and Tupy).
Cane diameter (indicate point of measurement):
   Floricane.—Base: 1.87 cm Midpoint: 1.55 cm Terminal: 0.68 cm.
   Inmature primocane.—Base: 1.32 cm Midpoint: 0.84 cm Terminal: 0.44 cm.
   Mature primocane.—Base: 1.76 cm Midpoint: 1.09 cm Terminal: 0.54 cm.
   Internode length.—

Base: 12.57 cm Midpoint: 6.61 cm Terminal: 3.31 cm.
Thorn density/30 cm.—Base: 0 Midpoint: 0 Terminal: 0.
Primocane color.—Base: reddish brown mixed green texture Midpoint: wine red color mixed green texture Terminal: wine red color.
Floricane color.—Base: reddish brown Midpoint: reddish brown Terminal: reddish brown mixed green texture.
Date of primocane emergence.—Primocanes emerge during the first two weeks of October (in the southern Hemisphere at 32° 45′ S. Lat., 220 m elev.) and continue emerging until the second week of December.
Date of budbreak.—Vegetative budbreak occurs from the 10th to the 20th of September (in the Southern Hemisphere at 32° 45′ S. Lat., 220 m elev.).
Foliage:
General description: Leaves are pentafoliate with abaxial trichomes, soft to the touch. The adaxial surface of the leaves have fewer trichomes and are rugose to the touch. Leaflets are green in color, but the adaxial side is a darker shade of green than the abaxial side. The leaflets are doubly serrate. There are no spines on any part of the leaf. The petioles and petiolules are wine red in color.
   Leaves.—Width: 15.22 cm Length: 18.88 cm (including petiole) Number of leaflets: 5 a 7.
   Leaflet.—Width: 5.50 cm Length: 8.85 cm (including petiolules) Margin: Doubly Serrate Shape: Each leaflet is Ovate with acutely or broadly acuminate. Color: Base Adaxial: Green with the central vein of lighter green color. Base Abaxial: Green, but of a lighter shade than the adaxial side, with yellowish colored veins. Midpoint Adaxial: Green, veins of the same color and shade. Midpoint Abaxial: Green, but of a lighter shade than the adaxial side, yellowish colored veins. Terminal Adaxial: Green, with the veins being the same color and shade. Terminal Abaxial: Green, but a lighter shade than the Adaxial side, with the veins being the same color.
   Petioles.—Length: 8.92 cm Color: wine red color.
   Petiolules.—Length: 1-4 cm Color: Wine red color with small yellow spots.
Flowers:
   Primocane.—N/A (Primocanes have no flowers).
   Floricane.—Date of bloom: (Southern Hemisphere) 10% bloom: 15th of September 50% bloom: 25th of September Last bloom: First week of October.
   Petal color.—Pure White.
   Reproductive organs.—Stamenst: Erect, numerous. Pistils: Numerous Pollen: Fertile and abundant. Ovary: Superior.
   Flower diameter.—3-4 cm.
   Petal size.—Width: 1.2 cm Length: 1.5 cm.
   Average number flowers per cluster.—10.
   Average number of petals per flower.—6.
   Peduncle length.—3-4 cm.
   Peduncle color.—verde.
Fruit:
General description: Emilia fruits are large, sweet, without bitter aftertaste common to blackberries. There is little color change (reversion) in drupelets from black to red in postharvest cold storage and the fruits can be stored for a long time (up to seven days). There is little to no incidence of post-harvest decay or rot (during seven days of storage at 5° C.).
   Primocane.—N/A.
   Floricane.—Average first ripe date: 23rd of November (7 days before Tupy), the fruiting period lasts for 40 days. Size: Medium (6.1 grams on average). Diameter: Equator: 1.75 cm Base pole: 1.81 cm Terminal pole: 1.72 cm Length: 2.81 cm Shape: Oblong (Elongate). Drupelet size: Medium, 0.44 cm Seed size: Small Firmness: Firm Flavor: Sweet with no bitter aftertaste. Soluble solids: 12.6° brix pH: Not measured. Acidity: 0.75% Processed quality: Not evaluated. Uses: The primary use is for fresh market sales due to its excellent post-harvest behavior. Prickles: None.
   Typical market use.—Fresh Market.
   Keeping quality.—Good.
   Shipping quality.—Good.

1. A new and distinct cultivar of Blackberry plant named ‘Emilia’ as described and shown herein.
